(19) United States
(12) Patent Application Publication
(10) Pub. No.: US 2016/0143917 A1
(43) Pub. Date: May 26, 2016

(54) COMPOSITIONS AND METHODS FOR MODULATING HIV ACTIVATION

(71) Applicant: CASE WESTERN RESERVE UNIVERSITY, Cleveland, OH (US)

(72) Inventors: Jonathan Karn, Cleveland, OH (US); Biswajit Das, Cleveland, OH (US)

(21) Appl. No.: 14/905,539

(22) PCT Filed: Jul. 29, 2014
(86) PCT No.: PCT/US2014/048596

§ 371 Date: Jan. 15, 2016

Related U.S. Application Data
(60) Provisional application No. 61/859,573, filed on Jul. 29, 2013.

(51) Int. Cl. A61K 31/565; C12N 15/113; A61K 31/415; A61K 31/11; A61K 45/06; A61K 31/7088; A61K 31/185

(52) U.S. Cl. CPC A61K 31/565; A61K 31/7088; C12N 15/113; A61K 31/185; A61K 31/11; A61K 45/06; A61K 31/415; C12N 2310/14; C12N 2320/31

(57) ABSTRACT

A pharmaceutical composition for inducing reactivation of latent provirus in an HIV infected cell includes an ESR-1 antagonist or an ESR-1 coactivator antagonist and a pharmaceutically acceptable carrier.

### RELATED APPLICATION

This application claims priority from U.S. Provisional Application No. 61/859,573, filed Jul. 29, 2013, the subject matter of which is incorporated herein by reference in its entirety.

### GOVERNMENT FUNDING

This invention was made with government support under Grant No. DP1-DA028869 and amFAR 108301 awarded by The National Institutes of Health and amFar, the foundation for AIDS research. The United States government has certain rights to the invention.

### TECHNICAL FIELD

The present invention relates to the use of estrogen receptor and/or estrogen receptor coactivator protein agonists and/or antagonists to modulate HIV activation of latent pro-virus in HIV infected mammalian cells.

### BACKGROUND

Highly active antiretroviral therapies (HAARTs) fail to eradicate the HIV-1 virus due to persistence of the virus in a long-lived pool of latently infected cells residing primarily in the resting memory CD4⁺ T-cell population. While HAART reduces the viral load in many patients to levels below the current limits of detection, the rapid mutation rate of the HIV virus limits the efficacy of this therapy, rendering HAART ineffective in treating latent HIV infection. An important additional site of infection is the microglial cell and perivascular macrophage populations in the brain where activated HIV infection can lead to neurocognitive disorders even in the presence of HAART. HIV may also persist in other myeloid lineage cells and in hematopoietic stem cells. Eliminating the latent reservoir is particularly challenging since it is established during the earliest stages of the infection. The reservoir is typically found in long-lived cells and it is likely that the reservoir can be replenished during episodes of viremia or by homeostatic replacement of latently infected cells. Since intensification of antiviral regimens does not eradicate the latent pool from the infected host, there is a need to develop entirely novel forms of therapy. One approach for attacking the latent reservoir is to induce transcription of the latent provirus while continuing treatment with antiviral drugs—a “shock and kill” strategy. An alternative approach is to uncover pharmaceutical agents that prevent HIV reactivation from latency.

### SUMMARY

Embodiments described herein relate to compositions and methods of modulating activity, expression, replication, and/or transcription of latent HIV pro-virus in HIV infected mammalian cells. The compositions can include therapeutically effective amounts of agents that modulate ESR-1 functional activity when administered to an HIV infected mammalian cell. The ESR-1 functional activity can be inhibited using, for example, ESR-1 antagonists and/or ESR-1 coactivator antagonists, to induce latent HIV activity, expression, replication, and/or transcription in the HIV infected mammalian cells. Alternatively, the ESR-1 functional activity can be induced and/or promoted using, for example, ESR-1 agonists and/or ESR-1 coactivator agonists, to suppress HIV-1 activity, expression, replication, and/or transcription in the HIV infected mammalian cell.

In some embodiment, the composition can comprise a pharmaceutical composition that includes one or more ESR-1 antagonists and/or an ESR-1 coactivator antagonists and another activator of latent HIV expression, such as a complementary inducer of HIV transcription, e.g., vorinostat, TNF-α, a protein kinase C agonist, and a pharmaceutically acceptable carrier. The pharmaceutical composition can further include one or more antiviral agents.

Other embodiments described herein relate to methods for inducing activation of latent HIV provirus expression in an HIV infected cell. The methods can include contacting the cell with a therapeutically effective amount of an ESR-1 antagonist and/or an ESR-1 coactivator antagonist. The cell can also be contacted with another activator of latent HIV expression, such as a complementary inducer of HIV transcription, e.g., vorinostat, TNF-α, a protein kinase C agonist. In some aspects, the ESR-1 antagonist and/or the ESR-1 coactivator antagonist and the other activator of latent HIV expression synergistically enhance reactivation of latent HIV expression compared to either agent alone when contacting the HIV infected cell. In still other aspects, the cell can also be contacted with one or more antiviral agents to treat the HIV infection.

Still other embodiments relate to methods of treating HIV infections in a subject. The methods can include administering to the subject a therapeutically effective amount of an ESR-1 antagonist and/or an ESR-1 coactivator antagonist. Another activator of latent HIV expression, such as a complementary inducer of HIV transcription, e.g., vorinostat, TNF-α, a protein kinase C agonist, can also be administered to the subject in combination with the ESR-1 antagonist and/or an ESR-1 coactivator antagonist. In some aspects, the ESR-1 antagonist and/or the ESR-1 coactivator antagonist and the other activator of latent HIV expression can synergistically enhance reactivation of latent HIV expression compared to either agent alone when administered to an HIV infected cell. In still other aspects, one or more antiviral agents can also be administered in combination with the ESR-1 antagonist and/or the ESR-1 coactivator antagonist to treat the HIV infection.

Yet other embodiments relate to a method of treating HIV infection in a subject that includes administering to the subject a therapeutically effective amount of an ESR-1 agonist and/or an ESR-1 coactivator agonist. The therapeutically effective amount is an amount required to inhibit HIV transcription in a latent HIV infected CD4⁺ T cell of the subject. In still other aspects, one or more antiviral agents can also be administered in combination with the ESR-1 agonist and/or the ESR-1 coactivator agonist to treat the HIV infection.

### BRIEF DESCRIPTION OF THE DRAWINGS

FIGS. 1(A-B) illustrates histograms showing GFP expression in latently HIV provirus infected Jurkat (T-cell) clone 2D10 cells carrying a GFP-expressing provirus super-infected by: (A) scrambled shRNA control; or (B) a single shRNA to ESR-1.

FIGS. 2(A-D) illustrate histograms showing GFP expression in latently HIV provirus infected Jurkat (T-cell) clone 2D10 cells carrying a GFP-expressing provirus that are: (A) unstimulated cells; (B) exposed to 100 pg/ml TNFα; (C) exposed to 2.5 μM Fulvestrant (ICI-182780); or (D) exposed to 100 pg/ml TNFα and Fulvestrant (ICI-182780).

FIGS. 3(A-C) illustrate HIV Nef protein expression of latent HIV-1 infected Th17 primary T-cells infected primary T-cells that are: (A) untreated; (B) stimulated using antibodies to CD3/CD28; or (C) stimulated with Fulvestrant (ICI-182780).

FIGS. 4(A-C) illustrate histograms showing GFP expression in latently HIV provirus infected Jurkat (T-cell) clone 2D10 cells carrying a GFP-expressing provirus that are: (A) treated with a sub-optimum amount of a potent HDAC inhibitor, SAHA (250 nM); (B) weakly stimulated with 50 μM Fulvestrant; or (C) pre-treatment with the ESR-1 antagonist Fulvestrant (50 μM) for one hour and 250 nM SAHA.

FIGS. 5(A-D) illustrate histograms showing GFP expression in CHME5/HIV cells, a latently infected Fetal Microglia Cell line carrying a GFP-expressing provirus, that are: (A) unstimulated; (B) administered a suboptimal dose of TNFα (10 ng/ml); (C) administered 2.5 mM Fulvestrant; or (D) pre-treated with Fulvestrant and administered a suboptimal dose of TNFα.

FIGS. 6(A-C) illustrate histograms showing GFP expression in latently HIV provirus infected Jurkat (T-cell) clone 2D10 cells carrying a GFP-expressing provirus that are: (A) stimulated with 400 pg/ml TNFα resulted; (B) administered 100 μM ESR-1 agonist PPT; or (C) pretreated with PPT, a potent ESR-1 agonist, for one hour and 400 pg/ml TNFα.

FIGS. 7(A-C) illustrate histograms showing GFP expression in latently HIV provirus infected Jurkat (T-cell) clone 2D10 cells carrying a GFP-expressing provirus that are: (A) exposed to 1 μM SAHA; (B) exposed to 100 μM ESR-1 agonist PPT; or (C) pre-treated with ESR-1 agonist, PPT, and SAHA, a potent broad-spectrum HDAC inhibitor that is commonly used to re-activate latent proviruses.

FIGS. 8(A-B) illustrate histograms showing GFP expression in latently HIV provirus infected Jurkat (T-cell) clone 2D10 cells carrying a GFP-expressing provirus that are: (A) stimulated with 5 μM Gossypol, an antagonist of the steroid receptor co-activator-3 (SRC-3/NCOA3); or (B) costimulated with 5 μM Gossypol and sub-optimum amount of TNFα (100 pg/ml).

FIGS. 9(A-B) illustrate histograms showing GFP expression in CHME5/HIV cells, a latently infected Fetal Microglia Cell line carrying a GFP-expressing provirus, that are: (A) stimulated with Gossypol; or (B) costimulated with Gossypol and sub-optimum amount of TNFα (10 ng/ml).

FIGS. 10(A-D) illustrate histograms showing GFP expression in latently HIV provirus infected Jurkat (T-cell) clone 2D10 cells carrying a GFP-expressing provirus that are: (A) grown and maintained in phenol-red free media supplemented with 10% charcoal-stripped Fetal Bovine Serum (FBS), a condition that removes hormones from the media; (B) administered 400 pg/ml TNFα; (C) exposed to 2 ng/ml β-estradiol; or (D) exposed to 2 ng/ml β-estradiol and 400 pg/ml TNFα.

FIGS. 11(A-F) illustrate plots showing re-activation of provirus in latently infected primary T-cells by Tamoxifen and/or SAHA, Gossypol and/or SAHA.

FIGS. 12(A-F) illustrate histograms showing ESR-1 agonist Stilbestrol blocks latent pro-virus reactivation with TNFα and SAHA in 2D10 cell line.

### DETAILED DESCRIPTION

Unless specifically addressed herein, all terms used have the same meaning as would be understood by those of skilled in the art of the present invention. The following definitions will provide clarity with respect to the terms used in the specification and claims to describe the present invention.

As used herein, “agonist” refers to a biologically active ligand, which binds to its complementary biologically active receptor and activates the latter either to cause a biological response in the receptor or to enhance pre-existing biological activity of the receptor. “Antagonist” refers to a biologically active ligand, which binds to its complementary biologically active receptor and does not activate the latter to cause the natural biological response in the receptor or to reduce pre-existing biological activity of the receptor. Generally, the terms “antagonist(s)” and “agonist(s)” as used herein encompasses also derivatives of said antagonist(s).

As used herein, the terms “subject,” “patient,” “individual,” and “host” used interchangeably herein, refer to a mammal, including, but not limited to, murines, felines, simians, humans, mammalian farm animals, mammalian sport animals, and mammalian pets. The term includes mammals that are infected with as well as those that are susceptible to infection by an immunodeficiency virus. In certain embodiments, the term refers to a human infected with HIV.

As used herein, “HIV” is used herein to refer to the human immunodeficiency virus. It is recognized that the HIV virus is an example of a hyper-mutable retrovirus, having diverged into two major subtypes (HIV-1 and HIV-2), each of which has many subtypes.

As used herein, “LTR” in the context of HIV LTR means the Long Terminal Repeat, a sequence repeated at the 5′ and 3′ ends of the HIV genome, which consists of the enhancer and promoter regions for gene expression (U3 region), the RNA start site, and untranslated RNA sequences (RU5) such as the genomic repeat and polyadenylation sites.

As used herein, the term “viral infection” describes a diseased state in which a virus invades healthy cells, uses the cell's reproductive machinery to multiply or replicate and ultimately lyse the cell resulting in cell death, release of viral particles and the infection of other cells by the newly produced progeny viruses. Latent infection by certain viruses, e.g., HIV, is also a possible result of viral infection.

As used herein, “latency”, “latent”, “latently infected reservoir” or grammatical equivalents thereof refer to the integration of a viral genome or a integration of a partial viral genome within a host cell genome further characterized by (i) the undetectable level of non-spliced viral RNA (<500 copies RNA/ml by a commonly used PCR assay; Chun et al., 1997, Proc Natl Acad Sci USA, 94:13193-13197); (ii) absence of detectable viral production; or (iii) only about 10⁵ to 10⁶ latently infected CD4 memory T cells in a subject (Williams et al., 2004, J Biol Chem 279(40):42008-42017). “Latency” also means a concept describing (i) an asymptomatic clinical condition; (ii) the state of viral activity within a population of cells, or (iii) the down-regulation or absence of gene expression within an infected cell. “Latency” in the context of the viral life cycle can also refer to a virus' “lysogenic phase.” In contrast, a virus is in the “lytic” phase if the viral genomes are packaged into a capsid or other viral structure, ultimately leading to lysis of the host cell and release of newly packaged viruses into the host.

As used herein, “effective amount”, “effective dose”, sufficient amount”, “amount effective to”, “therapeutically effective amount” or grammatical equivalents thereof mean a dosage sufficient to produce a desired result, to ameliorate, or in some manner, reduce a symptom or stop or reverse progression of a condition. In some embodiments, the desired result is an increase in latent HIV expression. In other embodiments, the desired result is the partial or complete eradication of a latent HIV reservoir. In an alternative embodiment, the desired result is the promotion of or the continued maintenance of HIV provirus latency. Amelioration of a symptom of a particular condition by administration of a pharmaceutical composition described herein refers to any lessening, whether permanent or temporary, lasting or transit that can be associated with the administration of the pharmaceutical composition.

As used herein, the terms “eliminating”, “eradicating” or “purging” are used interchangeably.

As used herein, “activator of latent HIV expression” means any compound that can stimulate proviral latent DNA integrated into the genome of a host to begin transcription initiation, transcription elongation or replication and production of infectious virus and/or cell surface antigens, such as gp120 and/or gp41. In some embodiments, an activator of latent HIV expression has an additive or synergistic effect when co-administered with an ESR-1 antagonist or ESR-1 coactivator antagonist described herein. Specific examples of activators of latent HIV expression are provided herein.

As used herein, “reactivated,” “reactivation” or grammatical equivalents thereof, in the context of in vivo reactivated HIV, refers to an HIV that, after a period of latency, becomes transcriptionally active, and in many instances forms infectious viral particles. The term “reactivated,” as used herein in the context of in vitro reactivated HIV in a subject cell, refers to an HIV (e.g., a recombinant HIV) that, after a period of latency, becomes transcriptionally active, i.e., a functional Tat protein mediates transcription from a functional HIV promoter (e.g., a long terminal repeat promoter).

As used herein, “HAART” refers to a treatment for HIV infection which is a cocktail of anti-viral drugs known as Highly Active Anti-Retroviral Therapy. Typically, HAART includes two reverse transcriptase inhibitors and a protease inhibitor.

As used herein, “HDAC inhibitor” or “inhibitor of HDAC” encompasses any synthetic, recombinant, or naturally-occurring inhibitor, including any pharmaceutical salts or hydrates of such inhibitors, and any free acids, free bases, or other free forms of such inhibitors capable of inhibiting the activity of a histone deacetylase (HDAC). “Hydroxamic acid derivative,” as used herein, refers to the class of histone deacetylase inhibitors that are hydroxamic acid derivatives. Specific examples of inhibitors are provided herein.

As used herein, the term “iRNA agent,” refers to small nucleic acid molecules used for RNA interference (RNAi), such as short interfering RNA (siRNA), double-stranded RNA (dsRNA), microRNA (miRNA) and short hairpin RNA (shRNA) molecules. The iRNA agents can be unmodified or chemically-modified nucleic acid molecules. The iRNA agents can be chemically synthesized or expressed from a vector or enzymatically synthesized. The use of a chemically-modified iRNA agent can improve one or more properties of an iRNA agent through increased resistance to degradation, increased specificity to target moieties, improved cellular uptake, and the like.

As used herein, the term “antisense RNA,” refers to a nucleotide sequence that comprises a sequence substantially complementary to the whole or a part of an mRNA molecule and is capable of binding to the mRNA.

As used herein, the term “antibody”, is defined as an immunoglobulin that has specific binding sites to combine with an antigen.

Embodiments described herein relate to compositions and methods of modulating activity, expression, replication, and/or transcription of latent HIV pro-virus in an HIV infected mammalian cell, and to compositions and methods useful for the treatment of HIV in a subject. It was found that ESR-1 (or Estrogen receptor-α, ESRα) is an important repressor of HIV transcription. It is believed that ESR-1 binding to the long terminal repeat (LTR) sequence of HIV-1 results in the repression of HIV-1 transcription in mammalian cells. It was found that alteration ESR-1 activity by the use of ESR-1 antagonists or an ESR-1 coactivator antagonist or by the use of ESR-1 agonists or an ESR-1 coactivator agonist can be used, respectively, to either promote the reactivation of latent HIV provirus in latently infected cells or limit their responses to exogenous stimuli.

Compositions for modulating activity, expression, replication, and/or transcription of latent HIV pro-virus in an HIV infected mammalian cell can therefore include therapeutically effective amounts of agents that modulate ESR-1 level and/or bioactivity when administered to the HIV infected mammalian cell.

In some embodiments, the ESR-1 functional activity can be inhibited using, for example, ESR-1 antagonists and/or an ESR-1 coactivator antagonists, to induce latent HIV activity, expression, replication, and/or transcription in the HIV infected mammalian cells as well as activation or reactivation of latent provirus in the HIV infected cell. The ESR-1 and ESR-1 coactivator antagonists described herein can include any agent capable of inhibiting or decreasing the level and/or bioactivity of ESR-1 in the HIV infected cell. An agent that inhibits or reduces one or more of, the level and/or bioactivity and function of ESR-1 refers to a composition comprised of a substance that decreases and/or suppresses the biological and/or functional activity of ESR-1 to suppress HIV-1 activation and/or transcription. The biological or functional activity of ESR-1 can be suppressed, inhibited, and/or blocked in several ways including: direct inhibition of the activity of the ESR-1 (e.g., by using small molecules or peptidomimetics, dominant negative polypeptides); inhibition of genes that express the ESR-1 (e.g., by blocking the expression or activity of the genes and/or proteins); activation of genes and/or proteins that inhibit one or more of, the activity and function of ESR-1 (e.g., by increasing the expression or activity of the genes and/or proteins); inhibition of genes and/or proteins that are downstream mediators of ESR-1 (e.g., by blocking the expression and/or activity of the mediator genes and/or proteins); introduction of genes and/or proteins that negatively regulate one or more of, the activity and function of ESR-1 (e.g., by using recombinant gene expression vectors, recombinant viral vectors or recombinant polypeptides); or gene replacement with, for instance, a hypomorphic mutant of ESR-1 (e.g., by homologous recombination, over expression using recombinant gene expression or viral vectors, or mutagenesis). In certain embodiments, ESR-1 antagonists and ESR-1 coactivator antagonists described herein exhibit relatively low toxicity, permitting subjects to withstand treatment with these agents.

In some embodiments, the ESR-1 antagonists can include a selective estrogen receptor down-regulator (SERD) agent. As used herein, the term “selective estrogen receptor down-regulator” in the context of ESR-1 is an agent, which selectively binds to ESR-1 over ESRβ leading to a reduction in ESR-1 protein levels and degradation of ESR-1 in a latently infected cell and by those means prevents ESR-1 from exerting its biological actions, e.g., maintaining HIV proviral latency in the cell. In one embodiment, SERD is Fulvestrant (FASLODEX, AstraZeneca). Additional ESR-1 antagonists can include, but are not limited to, ZK-191703, SR16234, RW58668, GW5638, ICI 164,384, AZD4992, a non steroidal SERD such as CH4986399, CH4893237, diphenylfuran based compounds, and compounds from U.S. Pat. No. 7,018,994 and U.S. Pat. App. No.: 2009/0062246, the specific examples of which are incorporated herein by reference.

In other embodiments, the ESR-1 antagonist can include Tamoxifen, its active metabolites, 4-hydroxytamoxifen and/or N-desmethyl-4-hydroxytamoxifen, and/or known analogues and/or derivatives of Tamoxifen. Analogues and/or derivatives of Tamoxifen are described in, for example, U.S. Pat. Nos. 4,803,227, 5,192,525, 5,219,548, 5,446,203, 5,540,925, 5,904,930, 6,096,874, 6,172,263, 6,245,352, and 8,785,501, which are herein incorporated by reference in their entirety.

Other examples ESR-1 antagonists that can be used in the methods described herein are disclosed in U.S. Pat. Nos. 8,629,130, 8,653,072, 8,710,243, and 8,703,810, which are incorporated herein by reference in their entirety.

Still other ESR-1 antagonists can be identified and screened for potential induction of latent HIV activation by using cell based assays described in Examples 1 and 2. For example to determine whether a composition or potential ESR-1 antagonist can induce HIV-1 provirus re-activation in latently infected primary T-cells (Th17), the ESR-1 antagonist can be administered to latently HIV provirus infected Jurkat (T-cell) clone 2D10 cells carrying a GFP-expressing provirus. The level of GFP expression can then be measured to determine activation. In another example, the ESR-1 antagonist can be administered to the latently infected primary T-cells (Th17) and phosphorylation of serine 175 of CDK9 can be measured by flow cytometry analysis. Along with CDK9 pSer175, expression of viral protein Nef can also measured.

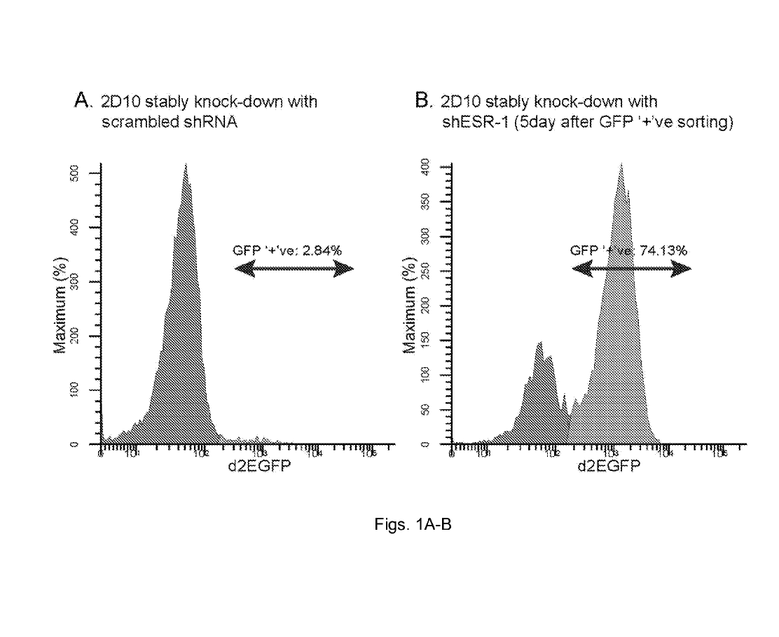

In other embodiments, the ESR-1 antagonist can include an agent, which reduces the expression of ESR-1 (e.g., ESR-1 iRNA agents, antisense RNA, vectors expressing iRNA agents or antisense RNA and the like). It was found that specific knock-down of the ESR-1 gene in latently infected human T-cells leads to constitutive re-activation of latent provirus (FIG. 1).

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro. The RNAi constructs may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of nitrogen or sulfur heteroatom. Modifications in RNA structure may be tailored to allow specific genetic inhibition while avoiding a general response to dsRNA. Likewise, bases may be modified to block the activity of adenosine deaminase. The RNAi construct may be produced enzymatically or by partial/total organic synthesis, any modified ribonucleotide can be introduced by in vitro enzymatic or organic synthesis.

Methods of chemically modifying RNA molecules can be adapted for modifying RNAi constructs (see, for example, Heidenreich et al. (1997) Nucleic Acids Res, 25:776-780; Wilson et al. (1994) J Mol Recog 7:89-98; Chen et al. (1995) Nucleic Acids Res 23:2661-2668; Hirschbein et al. (1997) Antisense Nucleic Acid Drug Dev 7:55-61). Merely to illustrate, the backbone of an RNAi construct can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides, α-configuration).

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications Inhibition is sequence-specific in that nucleotide sequences corresponding to the duplex region of the RNA are targeted for genetic inhibition.

In some embodiments, ESR-1 coactivator antagonists can include agents capable of modulating the expression or activity of a molecule that influences HIV transcription via their interaction with ESR-1. Exemplary ESR-1 interacting molecules are listed in Table 1 of the Example below and include AR, ATM, BCAR1, BRCA1, EP300, HIF1A, IGF1R, IRS1, NCOA1/SRC1, NCOA2/SRC2, NCOA3/SRC3, NRIP1, PELP1, PTPN1, RBBP8/RIM, RELA, SP1, SRC, TP53, and UIMC1. Therefore, in some embodiments, ESR-1 coactivator antagonists can include any agent capable of modulating the expression and/or activity of a compound listed in Table 1 to influence HIV transcription via their interaction with ESR-1. In certain embodiments, the ESR-1 coactivator antagonist is Gossypol (an antagonist of the steroid receptor co-activator-3 (SRC-3/NCOA3).

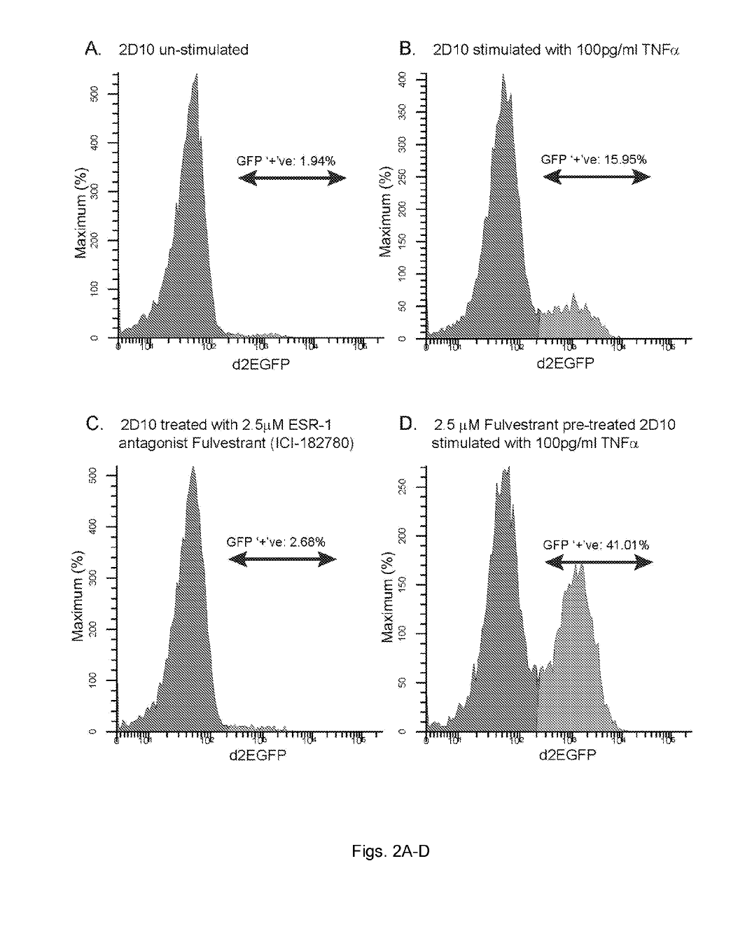

Additionally, as shown in FIGS. 2, 4, and 8, it was discovered that ESR-1 antagonists and ESR-1 coactivator antagonists can sensitize latently infected cells to sub-optimum dose concentrations of proviral activators. Accordingly, in some embodiments ESR-1 antagonists and/or ESR-1 coactivator antagonists can be administered in combination with activators of latent HIV expression to synergistically enhance reactivation of latently infected cell populations of cells compared to either agent alone.

In these embodiments, the ESR-1 antagonists and/or ESR-1 coactivator antagonists can be provided in a composition that can also include an activator of latent HIV expression that is not an ESR-1 antagonists and/or ESR-1 coactivator. Several activators of latent HIV expression can be used in the compositions and methods described herein. For example, an activator of latent HIV expression can include, but is not limited to, histone deacetylase (HDAC) inhibitors, protein kinase C agonists, and TNFα.

It has been demonstrated that HDAC inhibitors induce the transcriptional activation of the HIV-1 promoter. An HDAC inhibitor can be any molecule that effects a reduction in the activity of a histone deacetylase. This includes proteins, peptides, DNA molecules (including antisense), RNA molecules (including iRNA agents and antisense) and small molecules. In some embodiments of the present invention, a HDAC inhibitor is a small interfering RNA (siRNA), for example, a si/shRNA directed against HDAC1, 2, 3, 4, 5, 6, 7, 8, 9, 10, or 11. Non-limiting examples of such HDAC inhibitors are set forth below. It is understood that HDAC inhibitors include any salts, crystal structures, amorphous structures, hydrates, derivatives, metabolites, stereoisomers, structural isomers, and prodrugs of the HDAC inhibitors described herein.

In some embodiments, an HDAC inhibitor can include short-chain fatty acids (e.g., Sodium Butyrate, Isovalerate, Valerate, 4-Phenylbutyrate (4-PBA), Phenylbutyrate (PB), Propionate, Butyramide, Isobutyramide, Phenylacetate, 3-Bromopropionate, Tributyrin, Valproic acid (Vpa), Valproate, Valproate semisodium and pivaloyloxymethyl butyrate (PIVANEX)).

In other embodiments, an HDAC inhibitor can include a hydroxamic acid derivative (e.g., suberoylanilide hydroxamic acid (SAHA, vorinostat), Trichostatin analogs such as Trichostatin A (TSA) and Trichostatin C, m-Carboxycinnamic acid bishydroxamide (CBHA), Pyroxamide, Salicylbishydroxamic acid, Suberoyl bishydroxamic acid (SBHA), Azelaic bishydroxamic acid (ABHA) Azelaic-1-hydroxamate-9-anilide (AAHA), 6-(3-Chlorophenylureido) carpoic hydroxamic acid (3Cl-UCHA), Oxamflatin [(2E)-5-[3-[(phenylsulfonyl)amino]phenyl]-pent-2-en-4-ynohydroxamic acid], A-161906 Scriptaid, PXD-101 (Prolifix), LAQ-824, CHAP, MW2796, MW2996; or any of the hydroxamic acids disclosed in U.S. Pat. Nos. 5,369,108, 5,932,616, 5,700,811, 6,087,367, and 6,511,990). In certain embodiments, the HDAC inhibitor is SAHA.

In still other embodiments, an HDAC inhibitor can include benzamide derivatives (e.g., CI-994; MS-275 [N-(2-aminophenyl)-4-[N-(pyridin-3-yl methoxycarbonyl)aminomethyl]benzamide] and 3′-amino derivative of MS-275).

In yet other embodiments, an HDAC inhibitor can include cyclic peptides (e.g., Trapoxin A (TPX)-cyclic tetrapeptide (cyclo-(L-phenylalanyl-L-phenylalanyl-D-pipecolinyl-L-2-amino-8-oxo-9,10-epoxy decanoyl)), FR901228 (FK 228, depsipeptide), FR225497 cyclic tetrapeptide, Apicidin cyclic tetrapeptide [cyclo(N—O-methyl-L-tryptophanyl-L-isoleucinyl-D-pipecolinyl-L-2-amino-8-oxodecanoyl)], Apicidin Ia, Apicidin Ib, Apicidin Ic, Apicidin IIa, and Apicidin IIb, CHAP, HC-toxin cyclic tetrapeptide, WF27082 cyclic tetrapeptide, and Chlamydocin.

Additional HDAC inhibitors can include natural products, such as psammaplins and Depudecin, Electrophilic ketone derivatives such as Trifluoromethyl ketones, α-keto amides such as N-methyl-α-ketoamides, LSD1 polypeptide, TNF-alpha (TNFα), an inducible transcription factor NF-AT (nuclear factor of activated T cells), and Anti-IκBα or IκBε agents.

Protein kinase C (PKC) agonists can include non-tumor-promoting phorbol deoxyphorbol esters such as prostratin, the structural or functional analogs thereof described in US20120101283 A1, 12-deoxyphorbol 13-phenylacetate (DPP), Ingenol mebutate (ingenol-3-angelate, tradename PICATO) and bryostatins such as bryostatin-1.

The compositions described herein including an ESR-1 antagonist or ESR-1 coactivator antagonist, find use in a variety of methods. Methods described herein can be practiced in vitro and in vivo. In some embodiments, a composition including an ESR-1 antagonist or ESR-1 coactivator antagonist can be administered or contacted with a mammalian cell having a latent HIV infection. The administration can be in vivo, for example, by an intradermal, intravenous, subcutaneous, oral, aerosol, intramuscular and intraperitoneal route, or ex vivo, for example, by transfection, electroporation, microinjection, lipofection, adsorption, protoplast fusion, use of protein carrying agents, use of ion carrying agents, and use of detergents for cell permeabilization. In some embodiments, the ESR-1 antagonist or ESR-1 coactivator antagonist can be contacted with or administered to a mammalian cell in a human, preferably a human having a latent HIV infection.

Therefore, another aspect relates to a method of inducing activation of latent provirus expression in an HIV infected mammalian cell. The method includes contacting the mammalian cell with a composition that includes an amount of an ESR-1 antagonist or ESR-1 coactivator antagonist effective to activate latent HIV expression in the cell.

In some embodiments, the cell can also be contacted with or administered another activator of latent HIV expression. It was surprisingly found that an ESR-1 or an ESR-1 coactivator antagonist can synergize the effect that an activator of latent HIV expression has on the activation of latent HIV expression. Therefore, a lower dose of the activator of latent HIV expression can be used to essentially obtain the same or greater effect on activation of latent HIV expression than would be obtained when using the activator of latent HIV expression alone. Thus, in some embodiments, the effective amount of an activator of latent HIV expression, e.g., SAHA, administered to the mammalian cell is less than 50% of an amount of an activator of latent HIV expression that is required to obtain the same expression level in the absence of an ESR-1 or ESR-1 coactivator antagonist. In another embodiment the amount of an activator of latent HIV expression administered to the mammalian cell is less than 25%, preferably less than 20%, preferably less than 10%, more preferably less than 5% and even more preferably less than 2% of an amount an activator of latent HIV expression, that is required to obtain the same expression level in the absence of an ESR-1 antagonist. It is contemplated that using a much lower dose of, for example, an HDAC inhibitor, potentially avoids its toxicity at full dose.

In some embodiments, the ESR-1 antagonist and/or ESR-1 coactivator antagonist and the activator of latent HIV expression, which is not ESR-1 antagonist or ESR-1 coactivator antagonist, are administered simultaneously to the HIV infected cell. This can be done by contacting the mammalian cell with a composition comprising both compounds as described herein. In other embodiments, the activator of latent HIV expression and the ESR-1 antagonist or ESR-1 coactivator antagonist are administered to the HIV infected cell sequentially.

The methods described herein can be applied to any cell wherein an HIV genome is integrated into the cellular DNA. The cell can be a mammalian cell (e.g., a human cell). The cell can include a resting lymphoid mononuclear cell obtained from a mammal including e.g., lymphocytes, such as T cells (CD4, CD8, cytolytic, helper), B cells, natural killer cells; mononuclear phagocytes, such as monocytes, macrophages, epitheloid cells, giant cells, Kupffer cells, alveolar macrophages; dendritic cells, such as interdigitating dendrite cells, Langerhans cells, or follicular dendritic cells; granulocytes; etc. In certain embodiments, the cell is a CD4⁺ T cell, such as a resting memory CD4⁺ T-cell.

The ESR-1 antagonists and/or ESR-1 coactivator antagonists alone or in combination with the activators of latent HIV expression described herein, are also useful in the manufacture of pharmaceutical compositions. The pharmaceutical composition can include a therapeutically effective amount of the ESR-1 antagonists and/or ESR-1 coactivator antagonists alone or in combination with the activators of latent HIV expression along with excipients or carriers suitable for either enteral or parenteral administration to a subject. It is contemplated that a therapeutically effective amount of a pharmaceutical composition described herein can be administered to a subject for the treatment of, for example, latent HIV infection.

Therefore, in another aspect, a pharmaceutical composition described herein can be employed in a method for treating HIV latency in a subject. The subject can include a host latently infected with HIV, e.g., a human latently infected with HIV. The subject can include a subject having a persistent HIV reservoir despite treatment with antiretroviral therapy (e.g., HAART). Thus, in some embodiments, the therapeutically effective amount is the amount of a pharmaceutical composition to significantly decrease a latent HIV reservoir in a latently HIV infected subject.

In some embodiments, a therapeutically effective amount of a pharmaceutical composition including an ESR-1 antagonist and/or an ESR-1 coactivator antagonist can be administered to the latently HIV-infected subject. A pharmaceutical composition may include any combinations of ESR-1 antagonists, ESR-1 coactivator antagonists, and optionally activators of latent HIV expression compounds described herein along with a pharmaceutically acceptable carrier. In an exemplary embodiment, the ESR-1 antagonist is Fluvestrant and/or Tamoxifen. In another exemplary embodiment, the ESR-1 coactivator antagonist is Gossypol.

The pharmaceutical composition administered to the subject can optionally include an activator of latent HIV expression described above. In certain embodiments, the activator of latent HIV expression is selected from TNF-α and SAHA.

It is expected that a combination therapy including an ESR-1 antagonist or ESR-1 coactivator antagonist and one or more activators of latent HIV expression can purge the latent HIV from a subject's body since harboring cells with reactivated HIV can be recognized by specific CTLs (cytotoxic CD8+ T cells), by NK (Natural Killer) cells and by specific cytotoxic antibodies. It is also expected that a combination therapy described herein can purge the latent HIV from a subject's body by targeting and neutralizing the reactivated HIV-1 using anti-retroviral therapy, e.g., HAART.

Therefore, in some embodiments, a pharmaceutical composition administered to a subject includes a therapeutically effective amount of an ESR-1 antagonist or an ESR-1 coactivator antagonist, an activator of latent HIV expression, and another therapeutic agent useful in the treatment of HIV infection, such as a component used for HAART or immunotoxins.

As noted above, compositions described herein may be combined with one or more additional therapeutic agents useful in the treatment of HIV infection. It will be understood that the scope of combinations of the compounds of this invention with HIV/AIDS antivirals, immunomodulators, anti-infectives or vaccines is not limited to the following list, and includes in principle any combination with any pharmaceutical composition useful for the treatment of AIDS. The HIV/AIDS antivirals and other agents will typically be employed in these combinations in their conventional dosage ranges and regimens as reported in the art.

Examples of antiviral agents include (but not restricted) ANTIVIRALS Manufacturer (Tradename and/or Drug Name Location) Indication (Activity): abacavir GlaxoSmithKline HIV infection, AIDS, ARC GW 1592 (ZIAGEN) (nRTI); 1592U89 abacavir+GlaxoSmithKline HIV infection, AIDS, ARC (nnRTI); lamivudine+(TRIZIVIR) zidovudine acemannan Carrington Labs ARC (Irving, Tex.) ACH 126443 Achillion Pharm. HIV infections, AIDS, ARC (nucleoside reverse transcriptase inhibitor); acyclovir Burroughs Wellcome HIV infection, AIDS, ARC, in combination with AZT AD-439 Tanox Biosystems HIV infection, AIDS, ARC AD-519 Tanox Biosystems HIV infection, AIDS, ARC adefovir dipivoxil Gilead HIV infection, AIDS, ARC GS 840 (RTI); AL-721 Ethigen ARC, PGL, HIV positive, (Los Angeles, Calif.), AIDS alpha interferon GlaxoSmithKline Kaposi's sarcoma, HIV, in combination w/Retrovir AMD3100 AnorMed HIV infection, AIDS, ARC (CXCR4 antagonist); amprenavir GlaxoSmithKline HIV infection, AIDS, 141 W94 (AGENERASE) ARC (PI); GW 141 VX478 (Vertex) ansamycin Adria Laboratories ARC LM 427 (Dublin, Ohio) Erbamont (Stamford, Conn.) antibody which neutralizes; Advanced Biotherapy AIDS, ARC pH labile alpha aberrant Concepts (Rockville, Interferon Md.) AR177 Aronex Pharm HIV infection, AIDS, ARC atazanavir (BMS 232632) Bristol-Myers-Squibb HIV infection, AIDS, ARC (ZRIVADA) (PI); beta-fluoro-ddA Nat'l Cancer Institute AIDS-associated diseases BMS-232623 Bristol-Myers Squibb/HIV infection, AIDS, (CGP-73547) Novartis ARC (PI); BMS-234475 Bristol-Myers Squibb/HIV infection, AIDS, (CGP-61755) Novartis ARC (PI); capravirine Pfizer HIV infection, AIDS, (AG-1549, S-1153) ARC (nnRTI); CI-1012 Warner-Lambert HIV-1 infection cidofovir Gilead Science CMV retinitis, herpes, papillomavirus curdlan sulfate AJI Pharma USA HIV infection cytomegalovirus immune MedImmune CMV retinitis globin cytovene Syntex sight threatening CMV ganciclovir peripheral CMV retinitis delavirdine Pharmacia-Upjohn HIV infection, AIDS, (RESCRIPTOR) ARC (nnRTI); dextran Sulfate Ueno Fine Chem. Ind. AIDS, ARC, HIV Ltd. (Osaka, Japan) positive asymptomatic ddC Hoffman-La Roche HIV infection, AIDS, ARC (zalcitabine, (HIVID) (nRTI); dideoxycytidine ddl Bristol-Myers Squibb HIV infection, AIDS, ARC; Dideoxyinosine (VIDEX) combination with AZT/d4T (nRTI) DPC 681 & DPC 684 DuPont HIV infection, AIDS, ARC (PI) DPC 961 & DPC 083 DuPont HIV infection AIDS, ARC (nnRTRI); emvirine Triangle Pharmaceuticals HIV infection, AIDS, ARC (COACTINON) (non-nucleoside reverse transcriptase inhibitor); EL10 Elan Corp, PLC HIV infection (Gainesville, Ga.) efavirenz DuPont HIV infection, AIDS, (DMP 266) (SUSTIVA) ARC (nnRTI); Merck (STOCRIN) famciclovir Smith Kline herpes zoster, herpes simplex emtricitabine Triangle Pharmaceuticals HIV infection, AIDS, ARC FTC (COVIRACIL) (nRTI); Emory University emvirine Triangle Pharmaceuticals HIV infection, AIDS, ARC (COACTINON) (non-nucleoside reverse transcriptase inhibitor); HBY097 Hoechst Marion Roussel HIV infection, AIDS, ARC (nnRTI); hypericin VIMRx Pharm. HIV infection, AIDS, ARC recombinant human; Triton Biosciences AIDS, Kaposi's sarcoma, interferon beta (Almeda, Calif.); ARC interferon alfa-n3 Interferon Sciences ARC, AIDS indinavir; Merck (CRIXIVAN) HIV infection, AIDS, ARC, asymptomatic HIV positive, also in combination with AZT/ddI/ddC (PI); ISIS 2922 ISIS Pharmaceuticals CMV retinitis JE2147/AG1776; Agouron HIV infection, AIDS, ARC (PI); KNI-272 Nat'l Cancer Institute HIV-assoc. diseases lamivudine; 3TC Glaxo Wellcome HIV infection, AIDS, (EPIVIR) ARC; also with AZT (nRTI); lobucavir Bristol-Myers Squibb CMV infection; lopinavir (ABT-378) Abbott HIV infection, AIDS, ARC (PI); lopinavir+ritonavir Abbott (KALETRA) HIV infection, AIDS, ARC (ABT-378/r) (PI); mozenavir AVID (Camden, N.J.) HIV infection, AIDS, ARC (DMP-450) (PI); nelfinavir Agouron HIV infection, AIDS, (VIRACEPT) ARC (PI); nevirapine Boeheringer HIV infection, AIDS, Ingleheim ARC (nnRTI); (VIRAMUNE) novapren Novaferon Labs, Inc. HIV inhibitor (Akron, Ohio); pentafusaide Trimeris HIV infection, AIDS, ARC T-20 (fusion inhibitor); peptide T Peninsula Labs AIDS octapeptide (Belmont, Calif.) sequence PRO 542 Progenics HIV infection, AIDS, ARC (attachment inhibitor); PRO 140 Progenics HIV infection, AIDS, ARC (CCR5 co-receptor inhibitor); trisodium Astra Pharm. Products, CMV retinitis, HIV infection, phosphonoformate Inc other CMV infections; PNU-140690 Pharmacia Upjohn HIV infection, AIDS, ARC (PI); probucol Vyrex HIV infection, AIDS; RBC-CD4Sheffield Med. Tech HIV infection, AIDS, (Houston Tex.) ARC; ritonavir Abbott HIV infection, AIDS, (ABT-538) (RITONAVIR) ARC (PI); saquinavir Hoffmann-LaRoche HIV infection, AIDS, (FORTOVASE) ARC (PI); stavudine d4T Bristol-Myers Squibb HIV infection, AIDS, ARC didehydrodeoxy-(ZERIT.) (nRTI); thymidine T-1249 Trimeris HIV infection, AIDS, ARC (fusion inhibitor); TAK-779 Takeda HIV infection, AIDS, ARC (injectable CCR5 receptor antagonist); tenofovir Gilead (VIREAD) HIV infection, AIDS, ARC (nRTI); tipranavir (PNU-140690) Boehringer Ingelheim HIV infection, AIDS, ARC (PI); TMC-120 & TMC-125 Tibotec HIV infections, AIDS, ARC (nnRTI); TMC-126 Tibotec HIV infection, AIDS, ARC (PI); valaciclovir GlaxoSmithKline genital HSV & CMV infections virazole Viratek/ICN (Costa asymptomatic HIV positive, ribavirin Mesa, Calif.) LAS, ARC; zidovudine; AZT GlaxoSmithKline HIV infection, AIDS, ARC, (RETROVIR) Kaposi's sarcoma in combination with other therapies (nRTI); [PI=protease inhibitor nnRTI=non-nucleoside reverse transcriptase inhibitor NRTI=nucleoside reverse transcriptase inhibitor].

The additional therapeutic agent may be used individually, sequentially, or in combination with one or more other such therapeutic agents described herein (e.g., a reverse transcriptase inhibitor used for HAART, a protease inhibitor used for HAART, and ESR-1 antagonists, an ESR-1 coactivator antagonist and/or an activator of latent HIV expression). Administration to a subject may be by the same or different route of administration or together in the same pharmaceutical formulation.

According to this embodiment, a composition comprising an ESR-1 antagonist and an activator of latent HIV expression may be coadministered with any HAART regimen or component thereof. The current standard of care using HAART is usually a combination of at least three nucleoside reverse transcriptase inhibitors and frequently includes a protease inhibitor, or alternatively a non-nucleoside reverse transcriptase inhibitor. Subjects who have low CD4⁺ cell counts or high plasma RNA levels may require more aggressive HAART. For subjects with relatively normal CD4⁺ cell counts and low to non-measurable levels of plasma HIV RNA over prolonged periods (i.e., slow or non-progressors) may require less aggressive HAART. For antiretroviral-naive subject who are treated with initial antiretroviral regimen, different combinations (or cocktails) of antiretroviral drugs can be used.

Thus, in some embodiments, a pharmaceutical composition comprising an ESR-1 or ESR-1 coactivator antagonist and an activator of latent HIV expression may be coadministered with a “cocktail” of nucleoside reverse transcriptase inhibitors, non-nucleoside HIV reverse transcriptase inhibitors, and protease inhibitors. For example, a pharmaceutical composition including an ESR-1 antagonist and an HDAC inhibitor may be coadministered with a cocktail of two nucleoside reverse transcriptase inhibitors (e.g., ZIDOVUDINE (AZT) and LAMIVUDINE (3TC)), and one protease inhibitor (e.g., INDINAVIR (MK-639)). A pharmaceutical composition including an ESR-1 or ESR-1 coactivator antagonist and an activator of latent HIV expression, such as an HDAC inhibitor, may also be coadministered with a cocktail of one nucleoside reverse transcriptase inhibitor (e.g., STAVUDINE (d4T)), one non-nucleoside reverse transcriptase inhibitor (e.g., NEVIRAPINE (BI-RG-587)), and one protease inhibitor (e.g., NELFINAVIR (AG-1343)). Alternatively, a composition comprising an activator of latent HIV expression and an HDAC inhibitor may be coadministered with a cocktail of one nucleoside reverse transcriptase inhibitor (e.g., ZIDOVUDINE (AZT)), and two protease inhibitors (e.g., NELFINAVIR (AG-1343) and SAQINAVIR (Ro-31-8959)).

Coadministration in the context of this invention is defined to mean the administration of more than one therapeutic agent in the course of a coordinated treatment to achieve an improved clinical outcome. Such coadministration may also be coextensive, that is, occurring during overlapping periods of time.

This regimen may be continued for a period past the point when the levels of integrated and unintegrated HIV in active and memory T cells are undetectably low. At the end of the period, the subject is weaned from HAART and from the ESR-1 antagonist and activators of latent HIV expression. At this point, the subject is monitored for reestablishment of normal immune function and for signs of reemergence of HIV infection. Additionally, any needed conjunctive immunotherapy, such as bone marrow transplants, various cytokines or vaccination, may be administered. After this, the subject is monitored on a routine basis for life to detect reemergence of HIV infection, in which case repeat therapy according to the above embodiments may be performed.

Additionally, immunotoxins may be employed in a method of the present invention. In some embodiments, the administration of an ESR-1 antagonist or ESR-1 coactivator antagonist can render a cell having an integrated HIV genome sensitive to an immunotoxin. In some embodiments, an immunotoxin can be coadministered to a subject with an ESR-1 antagonist or ESR-1 coactivator antagonist and activators of latent HIV expression. An exemplary immunotoxin is an immunotoxin targeted to an HIV protein expressed on the exterior of cells, such as the viral envelope glycoprotein or a portion thereof. The term “immunotoxin” refers to a covalent or non-covalent linkage of a toxin to an antibody, such as an anti HIV envelope glycoprotein antibody. The toxin may be linked directly to the antibody, or indirectly through, for example, a linker molecule. The toxin can be selected from the group consisting of ricin-A and abrin-A.

Other embodiments described herein relate to compositions and methods effective in the treatment of HIV in a subject by inhibiting HIV reactivation in latently infected T-cells of a subject. It was discovered that selective ESR-1 agonists can be capable of suppressing TNF-α and SAHA HIV provirus reactivation in latently infected 2D10 Jurkat T cells.

In accordance with this aspect, a method of treating HIV infection in a subject can include administering to the subject a therapeutically effective amount of a pharmaceutical composition including an ESR-1 agonist or an ESR-1 coactivator agonist. The therapeutically effective amount is an amount effective to inhibit HIV transcription in the subject's latently infected T cells. In some embodiments, the T cell is a CD4⁺ T cell and the ESR-1 agonist or ESR-1 coactivator agonist is an agonist in T-cells.

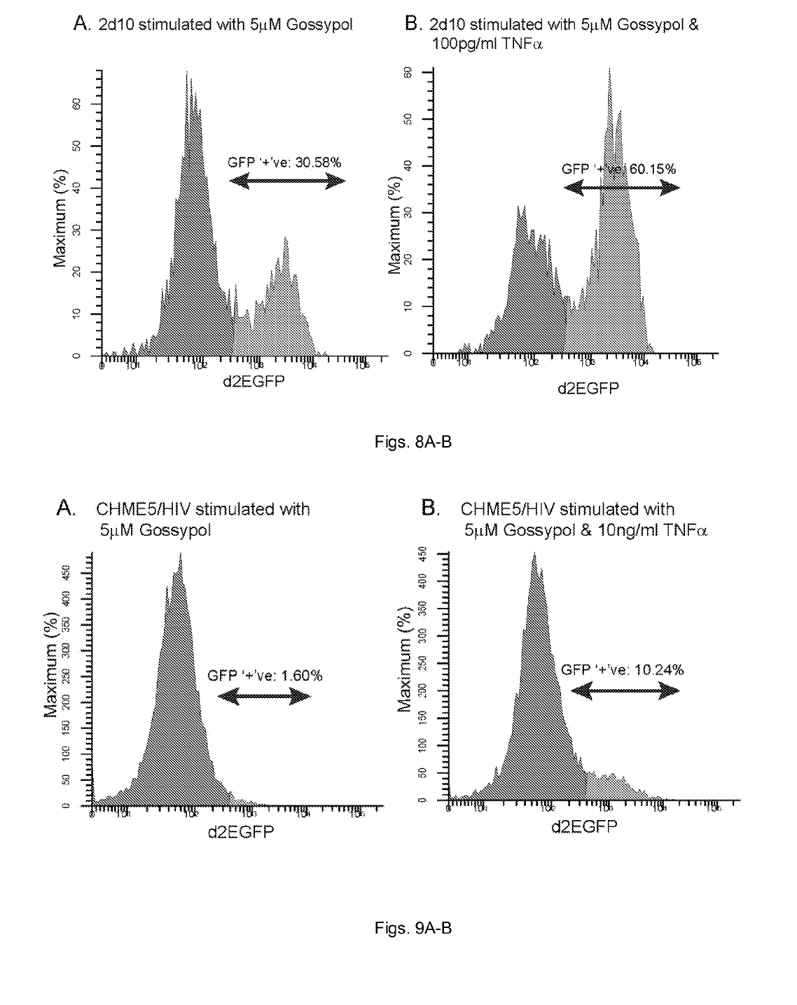

ESR-1 agonists can include a subtype-selective estrogen receptor agonist that displays selectivity for ESR-1 (ESRα) over ESRβ. Suitable Subtype-selective estrogen receptor agonists include propylpyrazole triol (PPT) (4,4′,4″-(4-Propyl-[1H]-pyrazole-1,3,5-triyl)trisphenol), SKF-82958 or a compound based on a triphenylfuran-scaffold. Mixed agonists having selectivity for ERα for use in the present invention can include triarylpyrazoles or etrahydroisoquinolines. In an exemplary embodiment illustrated in FIG. 8, the ESR-1 agonist is PPT.

ESR-1 coactivator agonists can include agents capable of modulating the expression or activity of a molecule that negatively influences HIV transcription and/or represses reactivation of latent HIV expression via their interaction with ESR-1. Examples of ESR-1 interacting molecules are listed in Table 1 of the Example below and include AR, ATM, BCAR1, BRCA1, EP300, HIF1A, IGF1R, IRS1, NCOA1/SRC1, NCOA2/SRC2, NCOA3/SRC3, NRIP1, PELP1, PTPN1, RBBP8/RIM, RELA, SP1, SRC, TP53, and UIMC1. Therefore, in some embodiments, ESR-1 coactivator agonists can include any agent capable of modulating the expression and/or activity of a compound listed in Table 1 and that negatively influences HIV transcription and/or represses reactivation of latent HIV expression via their interaction with ESR-1. In some embodiments, the ESR-1 coactivator agonist is a NCOA3/SRC3 agonist.

In some embodiments, the ESR-1 agonist or ESR-1 coactivator agonist can be administered to an HIV infected female, such as an HIV infected female before or during pregnancy, to inhibit HIV activation during pregnancy. The ESR-1 agonist or ESR-1 coactivator agonist can be administered to the female along with other known suppressor of HIV activation as well as retroviral therapies.

In some embodiments, the methods described herein, may optionally include the step of determining or detecting activation of latent HIV expression. Activation of latent HIV expression (also referred to as reactivation of latent HIV expression) results in the conversion of latently infected cells to productively infected cells. This transition can be measured by any characteristic of active viral infection, e.g., production of infectious particles, reverse transcriptase activity, secreted antigens, cell-surface antigens, soluble antigens, HIV RNA and HIV DNA, etc.

In one embodiment, such a method comprises determining or detecting an mRNA, e.g., an HIV mRNA. Other mRNAs, such as Tat mRNA, NF-κB mRNA, NF-AT mRNA and other mRNAs encoding polypeptides can also be determined using the well known methods including but not limited to hybridization and amplification based assays.

In another embodiment, amplification-based assays are used to measure the expression level of an HIV gene. In one embodiment, activation of latent HIV expression can be detecting by determining the expression level of an HIV polypeptide. The expression level of an HIV polypeptide may be determined by several methods, including, but not limited to, affinity capture, mass spectrometry, traditional immunoassays directed to HIV proteins (such as gp120 and reverse transcriptase), PAGE, Western Blotting, or HPLC as further described herein or as known by one of skill in the art.

Detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

In some embodiments, the compositions or pharmaceutical compositions described herein can be formulated by standard techniques using one or more physiologically acceptable carriers or excipients. Suitable pharmaceutical carriers are described herein and in “Remington's Pharmaceutical Sciences” by E. W. Martin. The small molecule compounds of the present invention and their physiologically acceptable salts and solvates can be formulated for administration by any suitable route, including via inhalation, topically, nasally, orally, parenterally, or rectally. Thus, the administration of the pharmaceutical composition may be made by intradermal, subdermal, intravenous, intramuscular, intranasal, intracerebral, intratracheal, intraarterial, intraperitoneal, intravesical, intrapleural, intracoronary or intratumoral injection, with a syringe or other devices. Transdermal administration is also contemplated, as are inhalation or aerosol administration. Tablets and capsules can be administered orally, rectally or vaginally.

For oral administration, a pharmaceutical composition or a medicament can take the form of, for example, a tablets or a capsule prepared by conventional means with a pharmaceutically acceptable excipient. Preferred are tablets and gelatin capsules comprising the active ingredient, i.e., a small molecule compound of the present invention, together with (a) diluents or fillers, e.g., lactose, dextrose, sucrose, mannitol, sorbitol, cellulose (e.g., ethyl cellulose, microcrystalline cellulose), glycine, pectin, polyacrylates and/or calcium hydrogen phosphate, calcium sulfate; (b) lubricants, e.g., silica, talcum, stearic acid, its magnesium or calcium salt, metallic stearates, colloidal silicon dioxide, hydrogenated vegetable oil, corn starch, sodium benzoate, sodium acetate and/or polyethyleneglycol; for tablets also (c) binders, e.g., magnesium aluminum silicate, starch paste, gelatin, tragacanth, methylcellulose, sodium carboxymethylcellulose, polyvinylpyrrolidone and/or hydroxypropyl methylcellulose; if desired (d) disintegrants, e.g., starches (e.g., potato starch or sodium starch), glycolate, agar, alginic acid or its sodium salt, or effervescent mixtures; (e) wetting agents, e.g., sodium lauryl sulphate, and/or (f) absorbents, colorants, flavors and sweeteners.

Tablets may be either film coated or enteric coated according to methods known in the art. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active compound.

The compositions described herein can also be formulated for parenteral administration by injection, for example by bolus injection or continuous infusion. Formulations for injection can be presented in unit dosage form, for example, in ampoules or in multi-dose containers, with an added preservative. Injectable compositions are preferably aqueous isotonic solutions or suspensions, and suppositories are preferably prepared from fatty emulsions or suspensions. The compositions may be sterilized and/or contain adjuvants, such as preserving, stabilizing, wetting or emulsifying agents, solution promoters, salts for regulating the osmotic pressure and/or buffers. Alternatively, the active ingredient can be in powder form for constitution with a suitable vehicle, for example, sterile pyrogen-free water, before use. In addition, they may also contain other therapeutically valuable substances. The compositions are prepared according to conventional mixing, granulating or coating methods, respectively, and contain about 0.1 to 75%, preferably about 1 to 50%, of the active ingredient.

For administration by inhalation, the compounds may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound and a suitable powder base, for example, lactose or starch.

Suitable formulations for transdermal application include an effective amount of a compound of the present invention with carrier. Preferred carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used.

Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

The compounds can also be formulated in rectal compositions, for example, suppositories or retention enemas, for example, containing conventional suppository bases, for example, cocoa butter or other glycerides.

Furthermore, the compounds can be formulated as a depot preparation. Such long-acting formulations can be administered by implantation (for example, subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

The compositions can, if desired, be presented in a pack or dispenser device that can contain one or more unit dosage forms containing the active ingredient. The pack can, for example, comprise metal or plastic foil, for example, a blister pack. The pack or dispenser device can be accompanied by instructions for administration.

In one embodiment, a pharmaceutical composition is administered to a subject, preferably a human, at a therapeutically effective dose to prevent, treat, or control a condition or disease as described herein, such as HIV latency.

The dosage of active compounds administered is dependent on the species of warm-blooded animal (mammal), the body weight, age, individual condition, surface area of the area to be treated and on the form of administration. The size of the dose also will be determined by the existence, nature, and extent of any adverse effects that accompany the administration of a particular small molecule compound in a particular subject. A unit dosage for oral administration to a mammal of about 50 to 70 kg may contain between about 5 and 500 mg of the active ingredient. Typically, a dosage of the active compounds of the present invention, is a dosage that is sufficient to achieve the desired effect. Optimal dosing schedules can be calculated from measurements of compound accumulation in the body of a subject. In general, dosage may be given once or more daily, weekly, or monthly. Persons of ordinary skill in the art can easily determine optimum dosages, dosing methodologies and repetition rates.

In another embodiment, a pharmaceutical composition including an ESR-1 antagonists or ESR-1 coactivator antagonist (or alternatively agonists thereof) is administered in a daily dose in the range from about 0.1 mg of each compound per kg of subject weight (0.1 mg/kg) to about 1 g/kg for multiple days. In another embodiment, the daily dose is a dose in the range of about 5 mg/kg to about 500 mg/kg. In yet another embodiment, the daily dose is about 10 mg/kg to about 250 mg/kg. In yet another embodiment, the daily dose is about 25 mg/kg to about 150 mg/kg. A preferred dose is about 10 mg/kg. The daily dose can be administered once per day or divided into subdoses and administered in multiple doses, e.g., twice, three times, or four times per day. However, as will be appreciated by a skilled artisan, activators of latent HIV expression and ESR-1 modulating agents may be administered in different amounts and at different times.

To achieve the desired therapeutic effect, compounds may be administered for multiple days at the therapeutically effective daily dose. Thus, therapeutically effective administration of compounds to treat a condition or disease described herein in a subject requires periodic (e.g., daily) administration that continues for a period ranging from three days to two weeks or longer. Typically, compounds will be administered for at least three consecutive days, often for at least five consecutive days, more often for at least ten, and sometimes for 20, 30, 40 or more consecutive days. While consecutive daily doses are a preferred route to achieve a therapeutically effective dose, a therapeutically beneficial effect can be achieved even if the compounds are not administered daily, so long as the administration is repeated frequently enough to maintain a therapeutically effective concentration of the compounds in the subject. For example, one can administer the compounds every other day, every third day, or, if higher dose ranges are employed and tolerated by the subject, once a week. A preferred dosing schedule, for example, is administering daily for a week, one week off and repeating this cycle dosing schedule for 3-4 cycles.

Optimum dosages, toxicity, and therapeutic efficacy of such compounds may vary depending on the relative potency of individual compounds and can be determined by standard pharmaceutical procedures in cell cultures or experimental animals, for example, by determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio, LD₅₀/ED₅₀. Compounds that exhibit large therapeutic indices are preferred. While compounds that exhibit toxic side effects can be used, care should be taken to design a delivery system that targets such compounds to the HIV infected cells to minimize potential damage to normal cells and, thereby, reduce side effects. In addition, combinations of compounds having synergistic effects described herein can be used to further reduce toxic side effects of one or more agents comprising a pharmaceutical composition of the invention.

The data obtained from, for example, cell culture assays and animal studies can be used to formulate a dosage range for use in humans. The dosage of such small molecule compounds lies preferably within a range of circulating concentrations that include the ED₅₀ with little or no toxicity. The dosage can vary within this range depending upon the dosage form employed and the route of administration. For any compounds used in the methods of the invention, the therapeutically effective dose can be estimated initially from cell culture assays. A dose can be formulated in animal models to achieve a circulating plasma concentration range that includes the IC₅₀ (the concentration of the test compound that achieves a half-maximal inhibition of symptoms) as determined in cell culture. Such information can be used to more accurately determine useful doses in humans. Levels in plasma can be measured, for example, by high performance liquid chromatography (HPLC). In general, the dose equivalent of compounds is from about 1 ng/kg to 100 mg/kg for a typical subject.

Following successful treatment, it may be desirable to have the subject undergo maintenance therapy to prevent the recurrence of the condition or disease treated.

Although the forgoing invention has been described in some detail by way of illustration and example for clarity and understanding, it will be readily apparent to one ordinary skill in the art in light of the teachings of this invention that certain variations, changes, modifications and substitution of equivalents may be made thereto without necessarily departing from the spirit and scope of this invention. As a result, the embodiments described herein are subject to various modifications, changes and the like, with the scope of this invention being determined solely by reference to the claims appended hereto. Those of skill in the art will readily recognize a variety of non-critical parameters that could be changed, altered or modified to yield essentially similar results.

The referenced patents, patent applications, and scientific literature, including accession numbers to GenBank database sequences, referred to herein are hereby incorporated by reference in their entirety as if each individual publication, patent or patent application were specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

As can be appreciated from the disclosure above, the present invention has a wide variety of applications. The invention is further illustrated by the following examples, which are only illustrative and are not intended to limit the definition and scope of the invention in any way.

### Example 1

We conducted studies using shRNA screens in order to identify and validate previously unidentified genes that are required to maintain HIV latency and/or play an essential role in HIV transcription. We performed genome-wide shRNA screening using a comprehensive shRNA library, combined with system biology classifications of the “hits”.

Jurkat T-cells (2D10) were super-infected with a synthetic shRNA library from Cellecta Inc. (Mountain View, Ca) containing a total of 82,500 shRNAs targeting 15,439 mRNA sequences. Cells carrying reactivated proviruses were then purified by sorting and the shRNA sequences identified by next-generation sequencing.

Ingenuity Pathway Analysis with top shRNA “hits” (>100,000 abundance value; top 10%) revealed that Estrogen Recptor-a (ESR-1) occupy a central position as a nodal molecule. All the input focus molecules in the IPA network remains as peripheral molecules and are directly influenced by ESR-1.

High hits identified in the STRING analysis include BCAR1, IRS1, SP1, NCOA1, NRIP1, UIMC1. We found that each of these molecules can influence HIV transcription via their interactions with ESR-1. As shown in Table 1, each of these molecules showed low abundance values in the GFP negative populations from the TNFα and SAHA screens, confirming that expression of those molecules contribute towards maintenance of pro-virus latency. shRNAs targeting RELA, BRCA1 and NCOA3, one of the upstream modulators of ESR-1, are present at relatively low abundance in the constitutively reactivated population and high abundance in the TNFα stimulated GFP negative population, indicating that these molecules make a positive contribution to HIV transcription.

TABLE 1
Abundance of shRNAs targeting ESR-1 interacting candidates

| | | 6 ng TNF | SAHA |
|---|---|---|---|
| Gene ID | GFP′ + ′ve | GFP′ − ′ve | GFP′ − ′ |
| AR | 3229 | 1523 | 5459 |
| ATM | 5407 | 9355 | 760 |
| BCAR1 | 12000 | 2276 | 128 |
| BRCA1 | 1231 | 27700 | 750 |
| EP300 | 5 | 4053 | 96 |
| ESR1 | 177161 | 5486 | 545 |
| HIF1A | 5274 | 6397 | 52116 |
| IGF1R | 2711 | 3067 | 27931 |
| IRS1 | 15877 | 4028 | 1158 |
| NCOA1/SRC1 | 22332 | 17272 | 8688 |
| NCOA2/SRC2 | 6947 | 7006 | 432 |
| NCOA3/SRC3 | 2511 | 36921 | 541 |
| NRIP1 | 22996 | 7684 | 447 |
| PELP1 | 3885 | 4536 | 489 |
| PTPN1 | 2 | 2133 | 1 |
| RBBP8/RIM | 1403 | 10626 | 671 |
| RELA | 10659 | 811838 | 5379 |
| SP1 | 14536 | 6512 | 1919 |
| SRC | 4252 | 4254 | 6663 |
| TP53 | 3342 | 2702 | 7771 |
| UIMC1 | 29540 | 1225 | 2 |

Consistent with these results, specific knock-down of ESR-1 in 2D10 cells with shRNA constitutively re-activates the latent provirus (FIG. 1). A. 2D10 cells infected by scrambled shRNA control. B. 2D10 cells infected with a single shRNA to ESR-1. The GFP ‘+’ve population was isolated by cell sorting and evaluated after further growth for 5 days. 74.13% of the cells remained constitutively activated. These results confirm that specific knock-down of ESR-1 gene in the system leads to constitutive re-activation of latent pro-virus.

FIG. 2 illustrates the ESR1 antagonist Fulvestrant weakly stimulates 2D10 reactivation and sensitizes the cells to reactivation by sub-optimal concentrations of TNF-α. A. Unstimulated cells showing the low basal activation levels of approximately 2%. B. 2D10 showed sub-optimal re-activation (15.95%) after exposure to 100 pg/ml TNFα. C. Activation of cells with 2.5 μM Fulvestrant. There was a small but significant reactivation noted. D. Reactivation of latent population by 100 pg/ml TNFα increased considerably (41.01%) after one hour pre-treatment with a potent ESR-1 antagonist, Fulvestrant (ICI-182780). The sensitization of the latent population by Fulvestrant indicates that ESR-1 expression supports the latency maintenance program of the HIV-1 infected Jurkat cells.

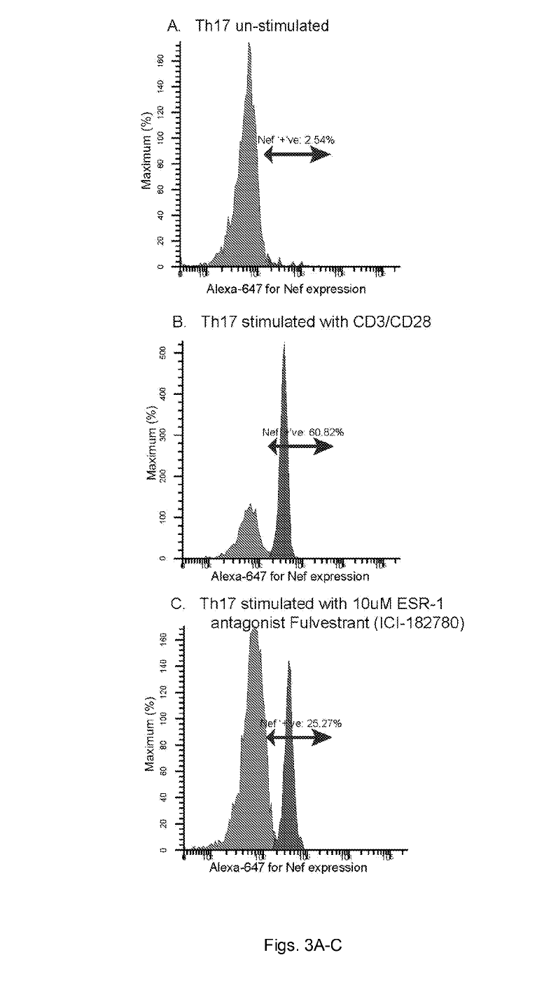

FIG. 3 illustrates latently infected primary T-cells are reactivated by the ESR1 antagonist, Fulvestrant (ICI.182780). A. Latently HIV-1 infected Th17 primary cells showed only 2.54% pro-virus expression, as measured by the expression the HIV Nef protein. B. Stimulation of the latently infected cells through the T-cell receptor using antibodies to CD3/CD28 re-activated 60.82% of the cells in the population. C. Downregulation of ESR-1 expression with its antagonist, Fulvestrant, reactivated 25.27% of the latent proviruses. Method: Naive T-cells from healthy donors were activated using anti-CD3/anti-CD18 antibodies and polarized in the direction of primary Th-17 cells using a cocktail of cytokines. The exponentially growing cells were infected with HIV-1 virus containing d2EGFP fused to CD8a for surface expression of the reporter gene. The CD8a positive cells were purified using CD8a antibody conjugated magnetic beads. The HIV-1 infected cells were allowed to become quiescent by culturing in media containing minimal levels of IL2.

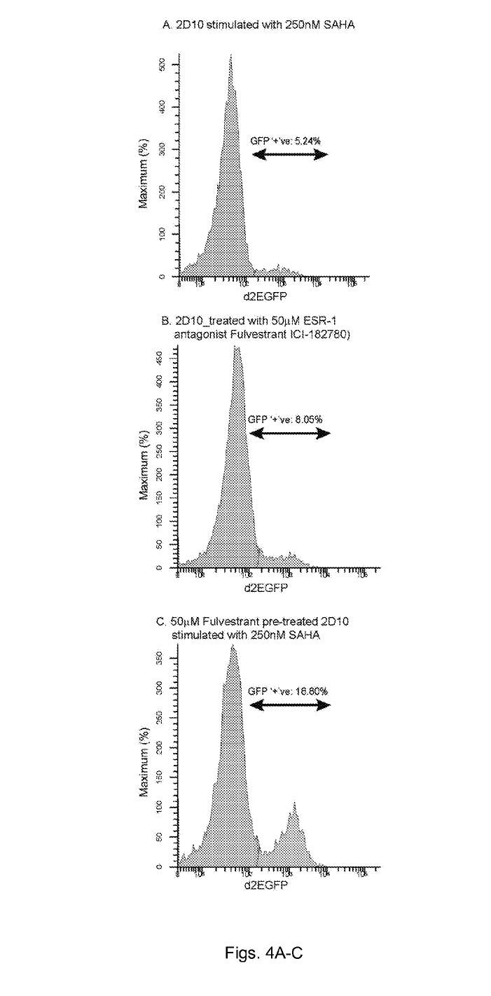

FIG. 4 illustrates the ESR-1 antagonist Fulvestrant weakly stimulates 2D10 reactivation and sensitizes the cells to reactivation by sub-optimal concentrations of the HDAC inhibitor SAHA. A. A sub-optimum amount of a potent HDAC inhibitor, SAHA (250 nM) induces 5.25% of the cells. B. 50 μM Fulvestrant weakly stimulated 2D10 cells raising GFP levels to 8.05%. C. Pre-treatment with the ESR-1 antagonist Fulvestrant (50 μM) for one hour increase the reactivation pro-virus by 250 nM SAHA to 18.80%. HDAC inhibitors such as SAHA are widely used as molecules to induce the pro-virus re-activation but typically these compounds are less efficient activators than TNFα in Jurkat cells or T-cell receptor stimulation in primary cells. The combined effects of ESR-1 antagonists and HDAC inhibitors may therefore allow more efficient reactivation of latently infected cell populations.

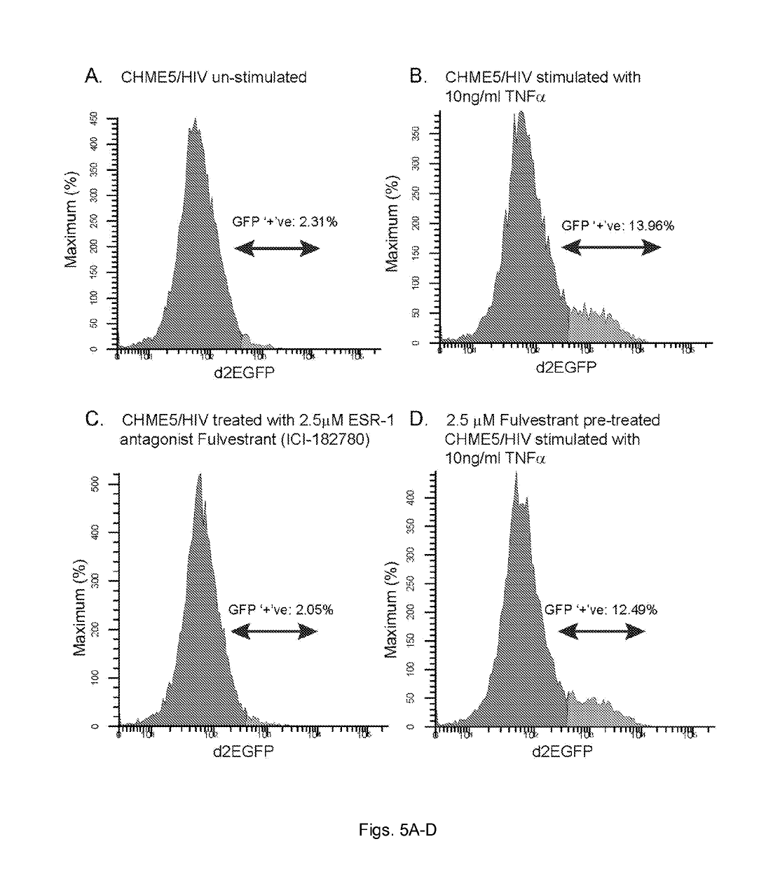

FIG. 5 illustrates latent HIV proviruses in microglial cells are not reactivated by the ESR-1 antagonist Fulvestrant. A. Unstimulated CHME5/HIV cells, a latently infected Fetal Microglia Cell line. Basal activation levels were 2.31%. B. Reactivation of CHME5/HIV cells with a suboptimal dose of TNFα (10 ng/ml) induced 13.96% of the proviruses. C. 2.5 mM Fulvestrant did not stimulated CHME5/HIV cells beyond basal levels. D. Pre-treatment with ESR-1 antagonist, Fulvestrant or ICI-182780 did not sensitize the latent population towards TNFα stimulation. These observations indicate that pro-virus reactivation by modulating the ESR-1 is specific for T-cell.

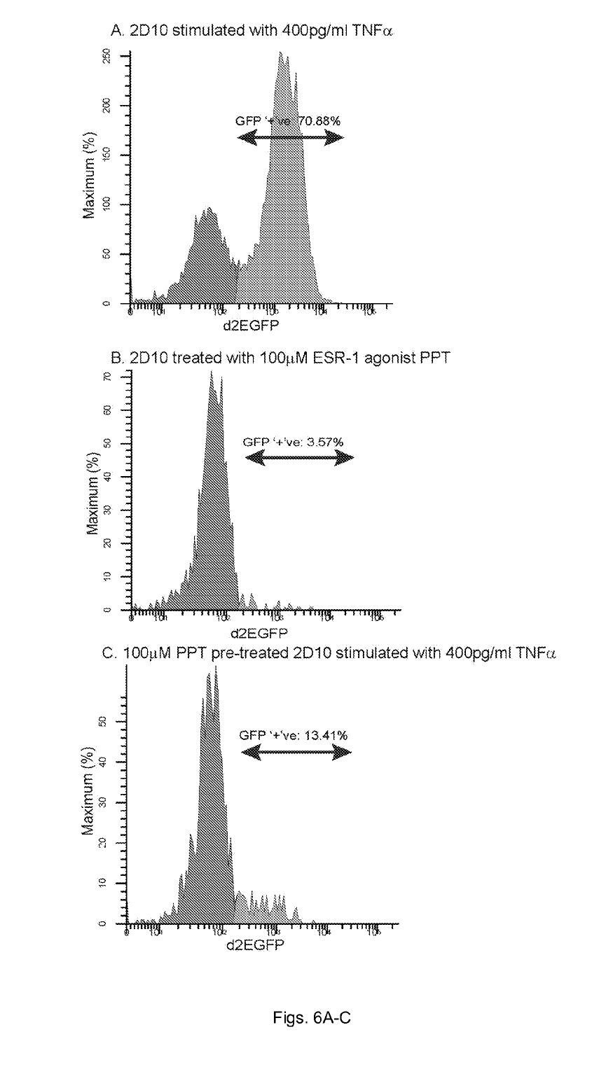

FIG. 6 illustrates the inhibition of HIV reactivation by TNFα by the ESR-1 agonist PPT. A. Latently infected 2D10 cells stimulated by 400 pg/ml TNFα resulted in a high level (70.88%) of proviral re-activation. B. Latently infected cells were not reactivated by exposure to 100 μM ESR-1 agonist PPT, C. Pretreatment of 2D10 cells with PPT, a potent ESR-1 agonist, for one hour decreased the reactivation induced by 400 pg/ml TNFα to only 13.41%. Forcing over-expression of ESR-1 by using its agonist blocks HIV transcription and prevents TNFα from inducing latent proviruses. This observation is consistent with the idea that a higher level of ESR-1 binding to the HIV LTR induced by the agonist results potent repression of HIV transcription.

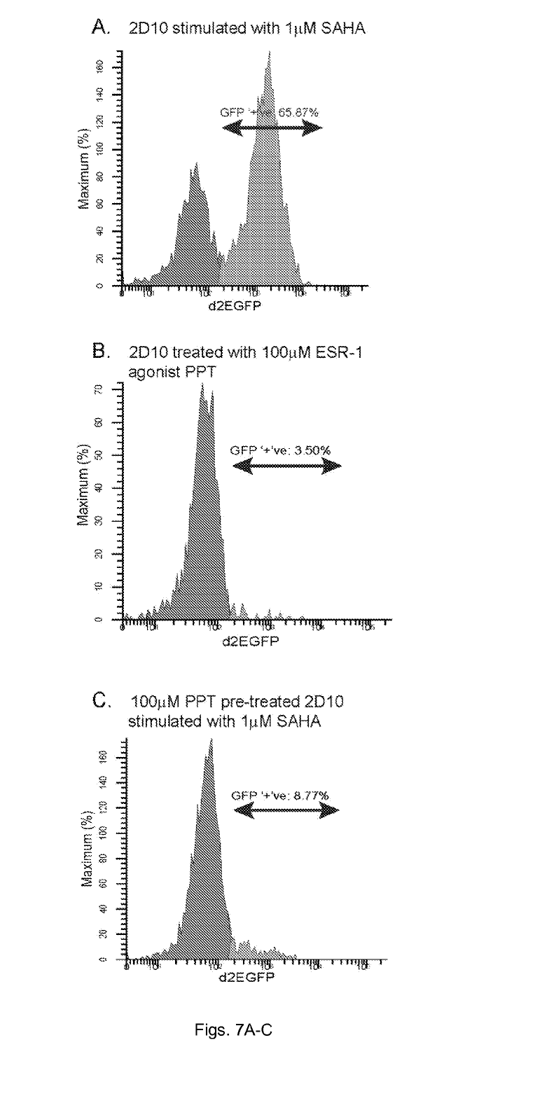

FIG. 7 illustrates the inhibition of HIV reactivation by SAHA by the ESR-1 agonist PPT. A. 2D10 cells exposed to 1 μM SAHA showed re-activation of 65.87% of the population. B. Latently infected cells were not reactivated by exposure to 100 μM ESR-1 agonist PPT. C. Pre-treatment with ESR-1 agonist, PPT, blocks the re-activation induced by SAHA resulting in activation of only 8.77% of the cells. SAHA is a potent broad-spectrum HDAC inhibitor that is commonly used to re-activate latent proviruses. Since the ERS-1 agonist PPT is able to block HIV reactivation induced by both SAHA and TNFα it is acting a general step in HIV transcription.

FIG. 8 illustrates 2D10 cells are stimulated by Gossypol, an antagonist of the steroid receptor co-activator-3 (SRC-3/NCOA3). A. Stimulation of 2D10 cells with 5 μM Gossypol induced 30.58% of proviral re-activation. B. Combined stimulation of 2D10 cells with 5 μM Gossypol and sub-optimum amount of TNFα (100 pg/ml) increase the re-activation to 60.15%). SRC-3 is an upstream activator of ESR-1. Blocking of active site of SRC-3 by Gossypol decreases ESR-1 activity. Elevated level of pro-virus re-action is thus obtained by stimulating the latently infected 2D10 cells by using Gossypol alone or in combination with low dose of TNFα. This provides further evidence that decreased levels of ESR-1 expression induce latent proviruses and that regulators for ESR-1 can also be exploited as pharmacologic targets for proviral reactivation.

FIG. 9 illustrates latently infected microglial cells are not activated by Gossypol. A. Stimulation of latently infected CHME5/HIV cells with Gossypol, showed 1.60% of pro-virus re-activation. B. Combined stimulation of CHME5/HIV cells with Gossypol and sub-optimum amount of TNFα (10 ng/ml) did not increase the re-activation of pro-virus (10.24%) beyond TNFα alone. In contrast to 2D10 cells, latently infected CHME5/HIV cells do not respond to down-regulation of SRC-3, the upstream modulator of ESR-1. This provides additional evidence that proviral reactivation by modulating ESR-1 is T-cell type specific.

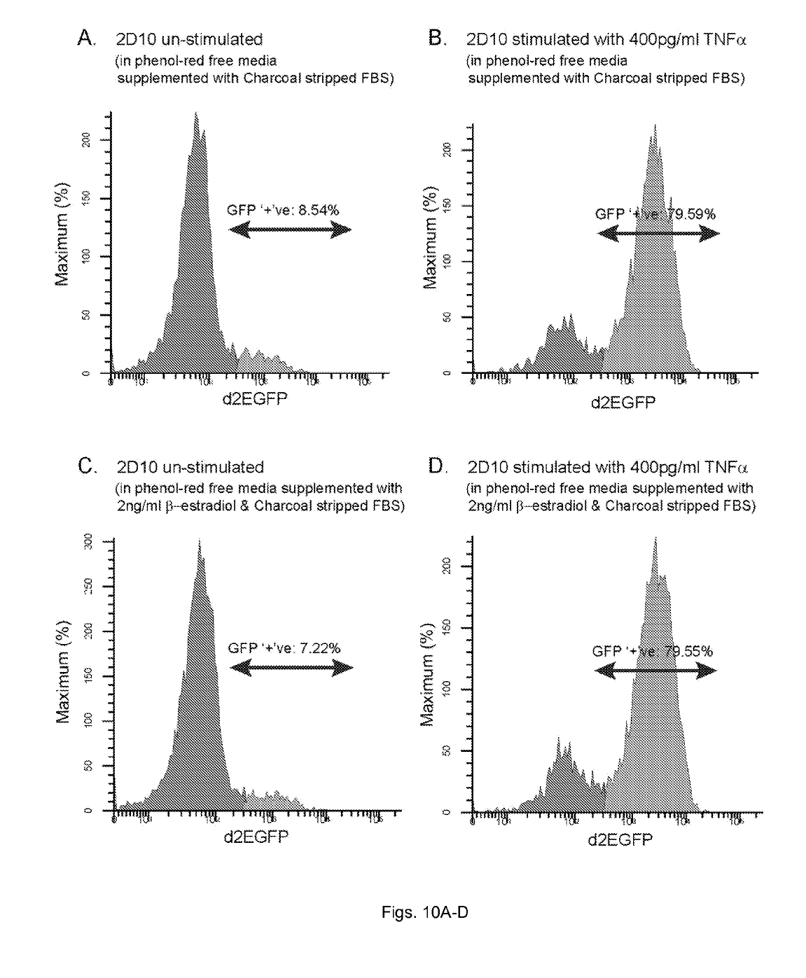

FIG. 10 illustrates estrogen in the media modestly represses HIV proviral expression. A. Latently infected 2D10 cells were grown and maintained in phenol-red free media supplemented with 10% charcoal-stripped Fetal Bovine Serum (FBS), a condition that removes hormones from the media. Under these conditions, un-stimulated 2D10 cells showed 8.54% provirus reactivation, which is higher than the 2% level seen in estrogen-containing media. B. 400 pg/ml TNFα stimulated 79.59% of the proviruses. C. Exogenous β-estradiol does not stimulate latent HIV proviruses. 2D10 cells in estrogen-depleted media exposed to 2 ng/ml β-estradiol show equivalent numbers of activated cells as estrogen-depleted 2D10 cells. D. 2 ng/ml β-estradiol did not enhance responsiveness to 400 pg/ml TNFα.

In summary, alteration of ESR-1 activity can be used to either promote the re-activation of latent proviruses or limit their response to exogenous stimuli. Thus, ESR-1 is a pharmacological target that can be exploited in the design of therapeutic strategies aimed at inducing HIV-1 proviral clearance via latent HIV reactivation or long-term silencing.

### Example 2

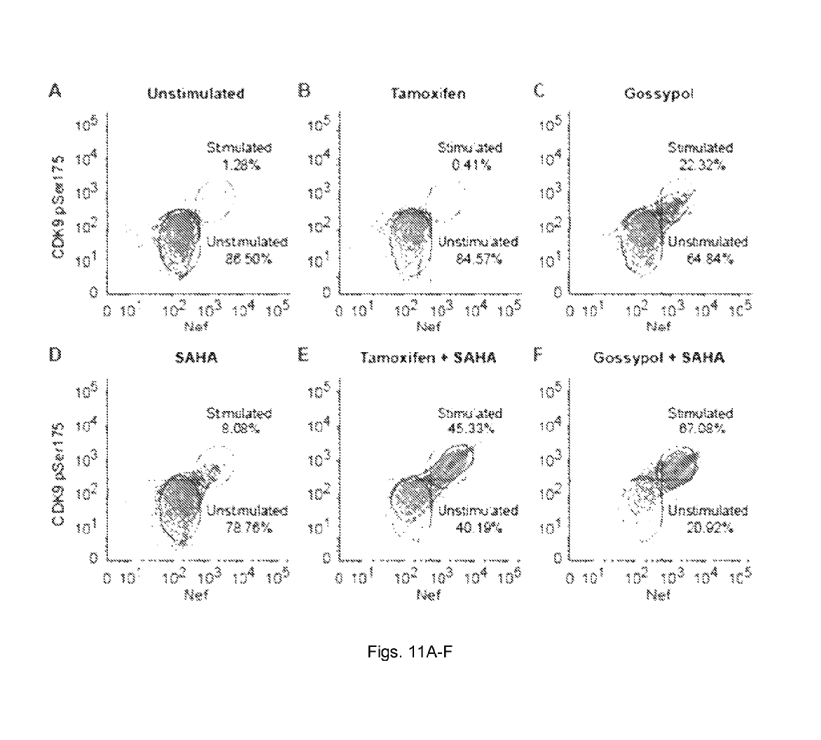

At the time of HIV-1 provirus re-activation one of the key marker is phosphorylation of serine 175 amino acid residue of CDK9 (CDK9 pSer175). To determine the status of HIV-1 provirus re-activation in latently infected primary T-cells (Th17) by ESR-1 antagonist Tamoxifen, phosphorylation of serine 175 of CDK9 was measured by flow cytometry analysis. Along with CDK9 pSer175, expression of viral protein Nef was also measured. As evident in Jurkat T-cell line 2D10 that blocking of ESR-1 by its antagonist does not re-activate latent provirus considerably but sensitizes the provirus for re-activation by sub-optimum dose of HDAC inhibitor SAHA/Vorinostat. Keeping that in mind, latently infected primary T-cell was treated with tamoxifen and SAHA alone or in combination where sub-optimum dose of (250 nM) was used (FIGS. 11 B, D, E). In case of 2D10 cell We have also found that blocking of SRC-3, an ESR-1 upstream modulator, by its antagonist Gossypol constitutively re-activate latent provirus. Therefore re-activation of latent provirus in Gossypol and/or SAHA treated primary T-cell was also examined (FIGS. 11 C-D). We found that blocking of ESR-1 expression by Tamoxifen alone failed to re-activate latent provirus but sensitizes the provirus and reverse the latency maintenance set-up that leads to re-activation by Tamoxifen and SAHA as evident by elevated expression of both CDK9 pSer175 and Nef (FIG. 11). Similarly gossypol treated cells constitutively re-activate latent provirus as increase in both CDK9 pSer175 and Nef were observed (FIG. 11)

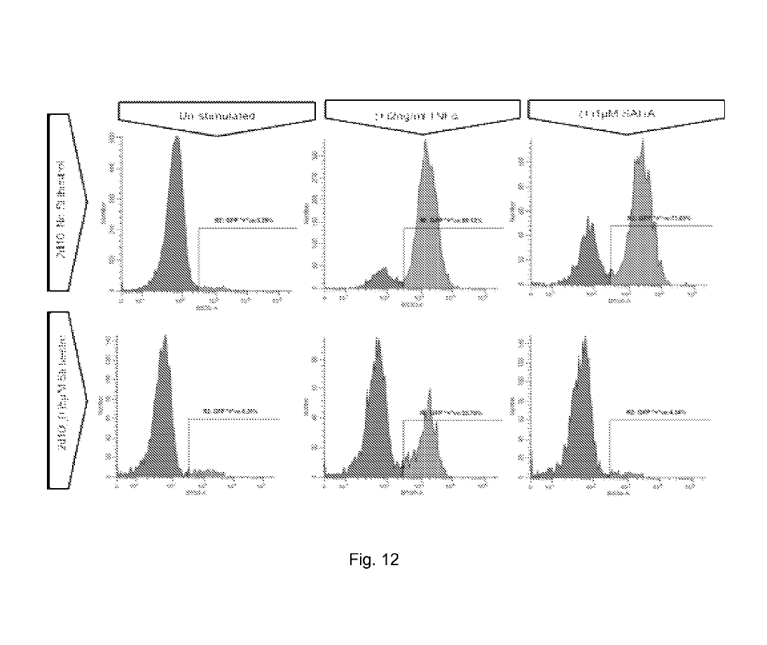

To further validate involvement of ESR-1 in latency maintenance program, we have examined a series of antagonists and agonist as represented in Table 2 on 2D10. In all the cases it was observed that blocking of ESR-1 by antagonist either constitutively re-activate or sensitizes the pro-virus to sub-optimum dose of TNFα (100 pg/ml) and SAHA (250 nM). It has also been found that pre-treatment with ESR-1 agonist block the provirus re-activation by optimum dose of TNFα (400 pg/ml) and SAHA (2 μM). Similarly pre-treatment of 2D10 by Stilbestrol (a potent ESR-1 agonist) block the re-activation of latent pro-virus with potent stimulator like TNFα and SAHA (FIG. 12)

TABLE 2

| | | | | |
|---|---|---|---|---|
| Un- | +100 pg/ml | +100 pg/ml | 250 nM | 250 nM |
| stimulated | TNFα | TNFα drug | SAHA | SAHA drug |

ESR-1 antagonists

| | | | | | |
|---|---|---|---|---|---|
| Control (2D10) | 2.79% | 16.15% | — | 14.34% | — |
| Fulvestrant (2.5 μM) | 2.68% | — | 41.01% | — | 17.78% |
| Y-134 (5 μM) | 17.77% | — | 63.13% | — | 44.16% |
| Endoxifen (5 μM) | 9.70% | — | 29.31% | — | 26.63% |
| MPP (5 μM) | 14.49% | — | 39.60% | — | 31.14% |
| Tamoxifen (10 μM) | 12.09% | — | 33.03% | — | 22.50% |

SRC3 inhibitor

| | | | | | |
|---|---|---|---|---|---|
| Gossypol (5 μM) | 30.58% | — | 60.15% | — | — |

| | | | | |
|---|---|---|---|---|
| Un- | 400 pg/ml | +400 pg/ml | 2 μM | 2 μM |
| stimulated | TNFα | TNFα drug | SAHA | SAHA drug |

ESR-1 agonist

| | | | | | |
|---|---|---|---|---|---|
| Control (2D10) | 2.63% | 70.88% | — | 65.43% | — |
| PPT (100 μM) | 3.57% | — | 13.41% | — | 6.76% |

ESR1 & 2 dual agonist

| | | | | | |
|---|---|---|---|---|---|
| Control (2D10) | 3.29% | 88.12% | — | 71.83% | — |
| Stilbestrol (5 μM) | 6.20% | — | 33.70% | — | 4.34% |

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1-15. (canceled)

16. A method for inducing activation of latent provirus expression in an HIV infected cell, the method comprising:
contacting the cell with an effective amount of a pharmaceutical composition, the pharmaceutical composition comprising an ESR-1 antagonist or an ESR-1 coactivator antagonist, an activator of latent HIV expression and a pharmaceutically acceptable carrier.

17. The method of claim 16, wherein the ESR-1 antagonist or the ESR-1 coactivator antagonist and the activator of latent HIV expression synergize to generate greater reactivation of latent HIV expression compared to either agent alone when contacted with the cell.

18. The method of claim 16, the HIV infected cell comprising a human CD4⁺ T cell.

19. The method of claim 16, the ESR-1 antagonist comprising a selective estrogen receptor down-regulator of ESR-1.

20. The method of claim 19, the selective estrogen receptor down-regulator of ESR-1 comprising Fulvestrant.

21. The method of claim 16, the ESR-1 antagonist comprising an ESR-1 shRNA.

22. The method of claim 16, the activator of latent HIV expression selected from an HDAC inhibitor and a protein kinase C agonist.

23. The method of claim 16, the HDAC inhibitor comprising a compound selected from the group consisting of a hydroxamic acid derivative, a short-chain fatty acid, a benzamide derivative, and a cyclic peptide.

24. The method of claim 23, the HDAC inhibitor comprising a hydroxamic acid derivative, wherein the hydroxamic acid derivative is suberoylanilide hydroxamic acid (SAHA).

25. The method of claim 22, the protein kinase C agonist comprising a compound selected from the group consisting of prostratin, 12-deoxyphorbol 13-phenylacetate (DPP), Ingenol mebutate, and a bryostatin.

26. The method of claim 16, wherein ESR-1 antagonist is Fulvestrant and the activator of latent HIV expression is suberoylanilide hydroxamic acid (SAHA).

27. The method of claim 16, the ESR-1 coactivator antagonist comprising Gossypol.

28. The method of claim 16, the pharmaceutical composition additionally comprising one or more antiviral agents.

29. The method of claim 28, the one or more antiviral agents comprising a component of HAART, the component of HAART selected from a nucleoside reverse transcriptase inhibitor, a non-nucleoside HIV reverse transcriptase inhibitor, and a protease inhibitor.

30. The method of claim 16, wherein the pharmaceutical composition fails to significantly activate the NF-kB signaling cascade in the HIV infected cell.

31. A method of treating HIV infection in a subject comprising:
administering to the subject a therapeutically effective amount of a pharmaceutical composition, the pharmaceutical composition comprising an ESR-1 antagonist or an ESR-1 coactivator antagonist, an activator of latent HIV expression, and a pharmaceutically acceptable carrier.

32. The method of claim 31, wherein the ESR-1 antagonist or the ESR-1 coactivator antagonist and the activator of latent HIV expression administered to the subject synergize to generate greater reactivation of latent HIV expression in an HIV infected cell of the subject compared to administration of either agent alone.

33. The method of claim 32, the HIV infected cell comprising a human CD4⁺ T cell.

34. The method of claim 31, the ESR-1 antagonist comprising a selective estrogen receptor down-regulator of ESR-1.

35. The method of claim 34, the selective estrogen receptor down-regulator of ESR-1 comprising Fulvestrant.

36. The method of claim 31, the ESR-1 antagonist comprising an ESR-1 shRNA.

37. The method of claim 31, the activator of latent HIV expression selected from an HDAC inhibitor and a protein kinase C agonist.

38. The method of claim 31, the HDAC inhibitor comprising a compound selected from the group consisting of a hydroxamic acid derivative, a short-chain fatty acid, a benzamide derivative, and a cyclic peptide.

39. The method of claim 38, the HDAC inhibitor comprising a hydroxamic acid derivative, wherein the hydroxamic acid derivative is suberoylanilide hydroxamic acid (SAHA).

40. The method of claim 37, the protein kinase C agonist comprising a compound selected from the group consisting of prostratin, 12-deoxyphorbol 13-phenylacetate (DPP), Ingenol mebutate, and a bryostatin.

41. The method of claim 31, wherein ESR-1 antagonist is Fulvestrant and the activator of latent HIV expression is suberoylanilide hydroxamic acid (SAHA).

42. The method of claim 31, the ESR-1 coactivator antagonist comprising Gossypol.

43. The method of claim 31, further comprising the step of administering to the subject a therapeutically effective amount of or more antiviral agents.

44. The method of claim 43, the one or more antiviral agents comprising a component of HAART, the component of HAART selected from a nucleoside reverse transcriptase inhibitor, a non-nucleoside HIV reverse transcriptase inhibitor, and a protease inhibitor.

45. The method of claim 31, the pharmaceutical composition additionally comprising one or more antiviral agents.

46. The method of claim 45, the one or more antiviral agents comprising a component of HAART, the component of HAART selected from a nucleoside reverse transcriptase inhibitor, a non-nucleoside HIV reverse transcriptase inhibitor, and a protease inhibitor.

47. The method of claim 31, wherein the pharmaceutical composition fails to significantly activate the NF-kB signaling cascade in the HIV infected cell.

48. A method of treating HIV infection in a subject comprising:
administering to the subject a therapeutically effective amount of a pharmaceutical composition, the pharmaceutical composition comprising an ESR-1 agonist or an ESR-1 coactivator agonist and a pharmaceutically acceptable carrier, wherein the therapeutically effective amount is the amount required to inhibit HIV transcription in a latent HIV infected CD4⁺ T cell of the subject.

49. The method of claim 48, the ESR-1 agonist comprising propylpyrazole triol (PPT).

50. The method of claim 48, further comprising the step of administering to the subject a therapeutically effective HAART regimen.
